(19) United States
(12) Patent Application Publication
(10) Pub. No.: US 2025/0345527 A1
(43) Pub. Date: Nov. 13, 2025

(54) Cap for medicament delivery device

(71) Applicant: SHL Medical AG, Zug (CH)

(72) Inventor: Nurettin Ali, Deerfield Beach, FL (US)

(21) Appl. No.: 18/993,047

(22) PCT Filed: May 26, 2023
(86) PCT No.: PCT/EP2023/064163

§ 371 Date: Jan. 10, 2025

Related U.S. Application Data
(60) Provisional application No. 63/392,212, filed on Jul. 26, 2022.

(30) Foreign Application Priority Data
Sep. 2, 2022 (EP) 22193737.8

(51) Int. Cl. A61M 5/32

(52) U.S. Cl. CPC A61M 5/3202; A61M 2207/00

(57) ABSTRACT

The present disclosure provides a cap for a medicament delivery device including a delivery member shield. The cap includes a body having a proximal end portion and a distal end portion, where the distal end portion has a smaller radial dimension than the proximal end portion. The distal end portion includes a central channel configured to receive the delivery member shield. The distal end portion further includes a plurality of flexible first clips and a plurality of second clips extending radially inwards into the central channel. Each second clip extends further in a proximal direction towards the proximal end portion than the first clips.

### CROSS-REFERENCE TO RELATED APPLICATIONS

The present application is a U.S. National Phase Application pursuant to 35 U.S.C. § 371 of International Application No. PCT/EP2023/064163 filed May 26, 2023, which claims priority to (i) U.S. Provisional Application No. 63/392,212 filed Jul. 26, 2022, and (ii) EP patent application Ser. No. 22/193,737.8 filed Sep. 2, 2022. The entire disclosure contents of these applications are herewith incorporated by reference into the present application.

### TECHNICAL FIELD

The present disclosure generally relates to medicament delivery devices.

### BACKGROUND

Medicament delivery devices, such as autoinjectors, may comprise a syringe that includes a needle which prior to use is covered by a needle shield. The needle shield may be covered by a front cap of the device. The cap is removed from the medicament delivery device prior to medicament administration. The cap may be designed to engage with the needle shield such that it grabs the needle shield when the cap is removed from the medicament delivery device.

### SUMMARY

A general object of the present disclosure is to provide a cap for a medicament delivery device that solves or at least mitigates the problems of the prior art.

There is hence according to a first aspect of the present disclosure provided a cap for a medicament delivery device including a delivery member shield, wherein the cap comprises: a body having a proximal end portion and a distal end portion, wherein the distal end portion has a smaller radial dimension than the proximal end portion, the distal end portion having a central channel configured to receive the delivery member shield, wherein the distal end portion comprises a plurality of flexible first clips and a plurality of second clips extending radially inwards into the central channel, wherein each second clip extends further in a proximal direction towards the proximal end portion than the first clips.

This design provides for an efficient removal of the delivery member shield from a delivery member such as a needle. In particular, the first clips, which have a shorter extension in the proximal direction than the second clips, may engage with the distal end of the delivery member shield, and the second clips, which reach further in the proximal direction, may engage with the outer surface or any recess or window provided in the perimeter surface of the delivery member shield. Thus, the first clips and second clips provide engagement with different features of a delivery member shield when the cap is pulled off from a medicament delivery device.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site.

Correspondingly, when the term “proximal direction” is used, this refers to the direction pointing towards the dose delivery site during use of the medicament delivery device.

When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component.

Similarly, “radial” or “radially” refer to a direction extending radially relative to the axis, and “rotation”, “rotational” and “rotationally” refer to rotation relative to the axis.

According to one embodiment each first clip is semi-circular in a circumferential direction of the cap.

According to one embodiment the second clips are pointy. This facilitates engagement with e.g., the outer surface, or a recess or window, provided in the outer surface of the delivery member shield.

According to one embodiment the second clips comprise two proximally directed tips. Each second clip may thus comprise two proximally directed tips. This makes each tip more mechanically flexible, which may facilitate engagement of the second clips with the delivery member shield.

According to one embodiment the tips of each second clip are separated by a notch.

According to one embodiment the distal end portion comprises distally extending arms, wherein each first clip is connected to two, in the circumferential direction of the cap, adjacent arms, and each second clip is connected to two, in the circumferential direction of the cap, adjacent arms.

According to one embodiment each arm is connected to one first clip and one second clip.

According to one embodiment the cap is a single integral part. Thus, the cap, including the first clips and the second clips, may be a single integral component. This simplifies the assembly process of a medicament delivery device as the cap itself is not required to be assembled from parts.

According to one embodiment the cap is made of plastic. Thus, also the first clips and the second clips may be made of plastic.

According to one embodiment each first clip has a first clip distal end and each second clip has a second clip distal end, wherein along a longitudinal axial direction of the cap, the first clip distal ends and the second clip distal ends are level.

There is according to a second aspect of the present disclosure provided a medicament delivery device comprising: a housing, a medicament container arranged in the housing, comprising a delivery member, a delivery member shield covering the delivery member, and a cap according to the first aspect, wherein the central channel of the distal end portion receives the delivery member shield.

According to one embodiment the first clips bear against a distal end of the delivery member shield.

According to one embodiment the delivery member shield comprises a plurality of radial shield windows, wherein the second clips each extend into a respective shield window of the plurality of radial shield windows.

The medicament delivery device may be an autoinjector.

According to a third aspect, there is provided a method of assembling a medicament delivery device according to the second aspect, comprising: a) attaching the cap to a proximal housing end of the housing, and b) arranging the medicament container in the housing via a distal opening of the housing, such that the delivery member shield is received by the distal end portion of the cap.

Generally, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 is a perspective view of an example of a cap for a medicament delivery device;

FIG. 2 is a longitudinal section of the cap in FIG. 1;

FIG. 3 shows a perspective view of the cap in FIG. 1 and a delivery member shield;

FIG. 4 shows the cap and delivery member shield in FIG. 3 with the delivery member shield arranged in the cap;

FIG. 5 is a perspective view of an example of a medicament delivery device; and

FIG. 6 shows an exploded view of the medicament delivery device in FIG. 5.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like elements throughout the description.

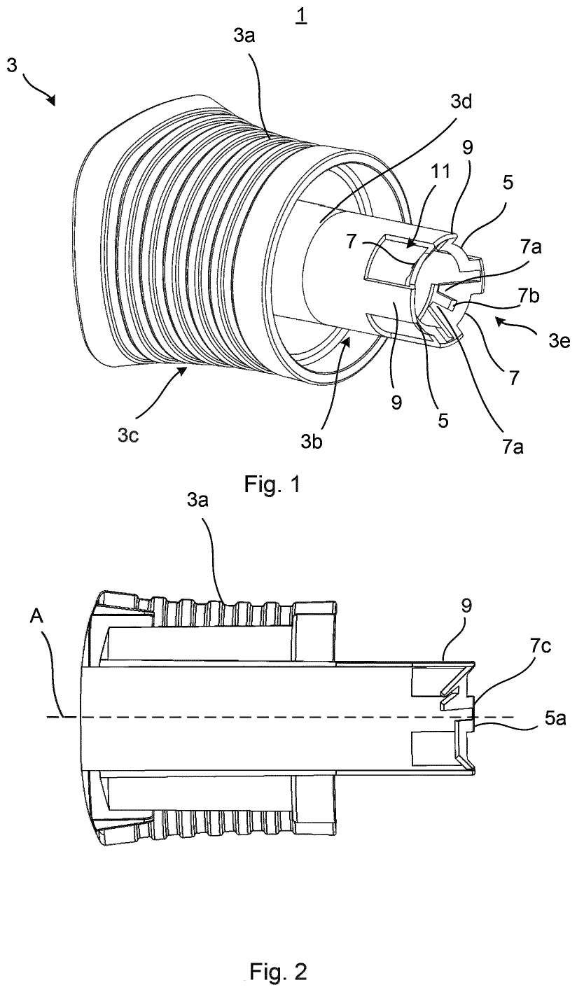

FIG. 1 shows an example of a cap 1 for a medicament delivery device, such as an autoinjector.

The cap 1 comprises a body 3. The body 3 has a proximal end portion 3a and a distal end portion 3b.

The proximal end portion 3a comprises an outer wall 3c. The outer wall 3c forms an outer surface of the cap 1.

The body 3 comprises an elongated structure 3d, extending concentrically with the outer wall 3c. The elongated structure 3d may be cylindrical or essentially cylindrical. The elongated structure 3d has a proximal portion that extends inside the outer wall 3c. The elongated structure 3d has a distal portion which defines the distal end portion 3b of the body 3. The distal end, and thus the distal end portion 3b protrudes from and extends distally relative to the proximal end portion 3a. The distal end portion 3b has a smaller radial dimension, or radius, than the proximal end portion 3a.

The distal end portion 3b is hollow. The distal end portion 3b comprises a central channel 3e extending axially along the central longitudinal axis of the cap 1. The central channel 3e extends all the way to the distal end of the distal end portion 3e, which is also the distal end of the cap 1. The central channel 3e is open at the distal end of the distal end portion 3b. The central channel 3e is configured to receive a delivery member shield of a medicament delivery device.

The distal end portion 3b comprises a plurality of radially flexible first clips 5 and a plurality of radially flexible second clips 7.

The first clips 5 extend radially inwards into the central channel 3e. The first clips 5 extend in a direction towards a proximal end of the cap 1.

The first clips 5 may be semi-circular in a circumferential direction of the cap 1.

The second clips 7 extend radially inwards into the central channel 3e. The second clips 7 extend in a direction towards a proximal end of the cap 1.

The second clips 7 may be pointy. Each second clip 7 may comprise two tips 7a which in the circumferential direction of the cap 1 are separated by a notch 7b.

According to the example, each first clip 5 faces another first clip 5. Each second clip 7 faces another second clip 7.

According to the example, the first clips 5 and the second clips 7 are arranged alternatingly in the circumferential direction of the cap 1.

The distal end portion 3b comprises a plurality of distally extending arms 9.

Each first clip 5 is connected to two adjacent arms 9. The arms 9 to which each first clip 5 is connected to are arranged adjacent to each other in the circumferential direction of the cap 1.

Each second clip 7 is connected to two adjacent arms 9. The arms 9 to which each second clip 7 is connected to are arranged adjacent to each other in the circumferential direction of the cap 1.

The first clips 5 and the second clips 7 form a distal end of the arms 9 and extend in the proximal direction from this position.

There is a radial cap window 11 between each pair of adjacent arms 9. Each radial cap window 11 may be arranged axially aligned with a respective one of the first clips 5 and second clips 7.

Turning now to FIG. 2, a longitudinal section of the cap 1 is shown. The first clips 5 and the second clips 7 define the distal end of the distal end portion 3b of the body 3. Each first clip 5 has a first clip distal end 5a and each second clip 7 has a second clip distal end 7c. The first clip distal ends 5a and the second clip distal ends 7c are axially level along the longitudinal axis A of the cap 1.

The second clips 7 extend further in the distal direction towards the distal end portion 3a of the body 3 than the first clips 5. Each second clip 7 thus has an axial extension which is longer than that of a first clip 5.

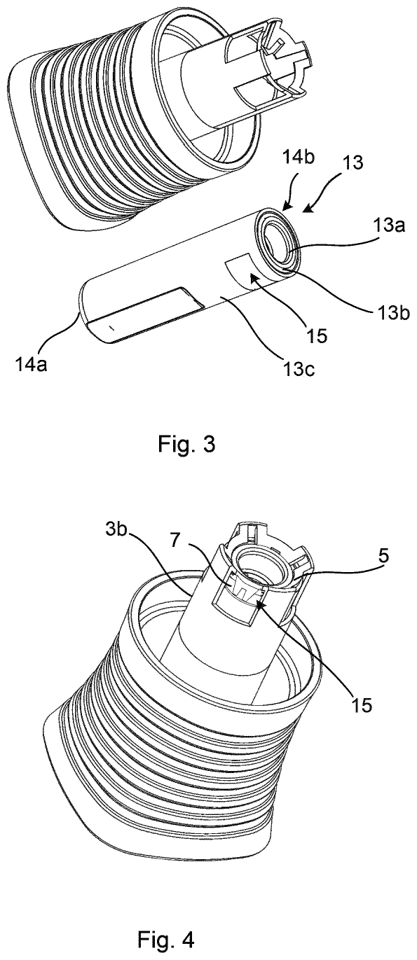

FIG. 3 shows the cap 1 and a delivery member shield 13. The delivery member shield 13 is according to the example configured to receive a needle of a syringe or medicament container. The delivery member shield 13 is thus according to the example a needle shield.

The delivery member shield 13 has a proximal end 14a and a distal end 14b.

The delivery member shield 13 comprises a flexible delivery member shield 13a, a rigid delivery member shield 13b, and a rigid outer cover 13c. The flexible delivery member shield 13a is arranged innermost, followed by the rigid delivery member shield 13b, followed by the rigid outer cover 13c, which is arranged outermost. The flexible delivery member shield 13a may be made of an elastomeric material and the rigid delivery member shield 13b and the rigid outer cover 13c may be made of crosslinked polymeric material, for example.

The rigid outer cover 13c has an outer surface provided with radial shield windows 15.

FIG. 4 shows when the delivery member shield 13 is arranged in the central channel 3e of the cap 1.

Each shield window 15 is aligned axially with a respective second clip 7. In particular, the tips 7a of each second clip 7 extend into a shield window 15.

The first clips 5 are arranged axially distally relative to the distal end 14b of the delivery member shield 13. The first clips 5 may bear against the distal end 14b of the delivery member shield 13.

When the cap 1 is pulled off a delivery member that extends into the delivery member shield 13, the first clips 5 engage with the distal end 14b of the delivery member shield 13 and the second clips 7 engage with a proximal edge of a respective one of the shield windows 15. The delivery member shield 13 is thus removed from the delivery member with the cap 1 efficiently.

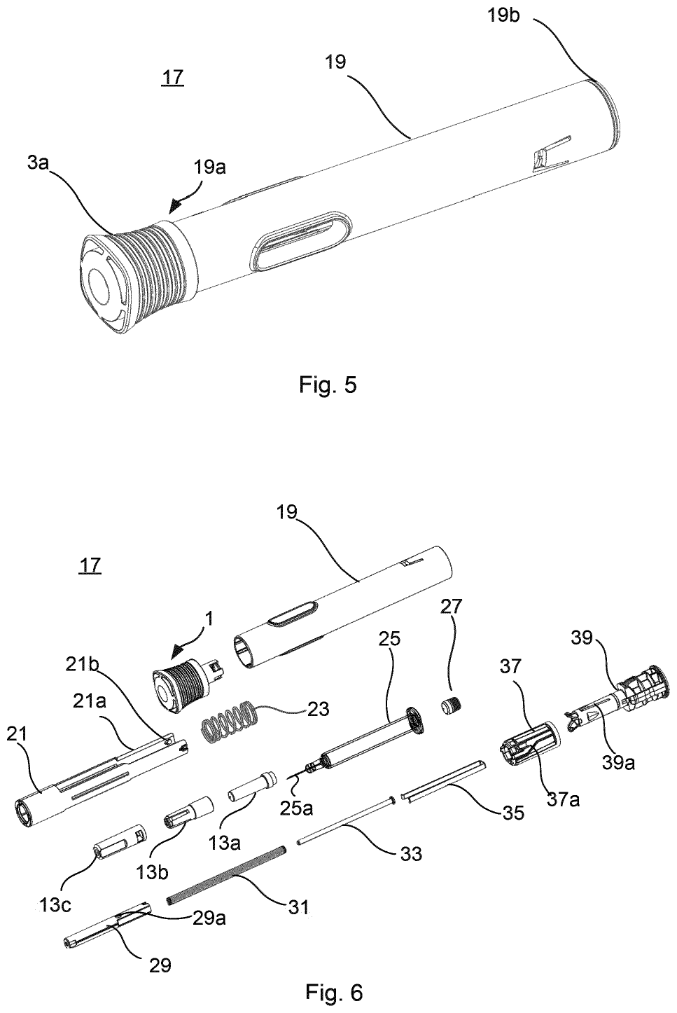

FIG. 5 shows an example of a medicament delivery device 17.

The medicament delivery device 17 comprises a housing 19. The housing 19 has a proximal housing end 19a and a distal housing end 19b.

The medicament delivery device 17 comprises the cap 1. The cap 1 forms a front end, or proximal end, of the medicament delivery device 17 prior to its removal. The proximal end portion 3a of the body 3 extends proximally from the proximal housing end 19a. The distal end portion 3b is received by the housing 19.

FIG. 6 shows an exploded view of the medicament delivery device 17 in FIG. 5. It is to be noted that the cap 1 may be used with a large number of different medicament delivery device designs and the one exemplified in FIGS. 5-6 is only described for illustrative purposes.

The medicament delivery device 17 comprises a delivery member cover 21 arranged in the housing 19 and configured to be moved between an extended position relative to the housing 19, in which it extends through an opening in the proximal housing end 19a, to a retracted position in which it is moved in the distal direction towards the distal housing end 19b.

The delivery member cover 21 is biased towards the extended position. The medicament delivery device 17 may thus comprise a first resilient member 23, such as a spring, configured to bias the delivery member cover 21 towards the extended position.

The delivery member cover 21 has proximally directed legs 21a, each provided with a radially inwards extending protrusion. 21b.

The medicament delivery device 17 comprises a medicament container provided 25 with a delivery member 25a. According to the example, the medicament container 25 is a syringe and the delivery member 25a is a needle.

The medicament delivery device 17 further comprises a stopper 27 arranged in the medicament container 25.

The medicament delivery device 17 also comprises a plunger rod 29, a second resilient member 31, a rod 33, a U-shaped bracket 35, a rotator 37, and a rear cap body 39. The components 29-39 form a power pack, which provides the mechanical power for medicament expulsion from the medicament container 25.

The plunger rod 29 is hollow, and comprises a distal end opening, in which the rod 33 is arranged, and in which the second resilient member 31 is arranged around the rod 33. The second resilient member 31 biases the plunger rod 29 in the proximal direction.

The U-shaped bracket 35 has proximally extending legs that extend radially outside of and along the plunger rod 29. It's function is to provide an audible click when the medicament administration commences and/or has reached its end.

Moreover, the plunger rod 29 has radial recesses 29a in its outer surface.

The rear cap body 39 has a proximal opening configured to receive the plunger rod 29. The rear cap body 39 has radially flexible arms 39a with radially inwards extending structures configured to engage with a respective one of the radial recesses 29a of the plunger rod 29. The rotator 37 is arranged around a proximal portion of the rear cap body 39, provided with the flexible arms 39a. The rotator 37 is initially in a first rotator position in which an inner surface of the rotator 37 prevents the flexible arms 39a to flex radially outwards. The plunger rod 29 is thus maintained in its initial axial position.

The rotator 37 is configured to be rotated from the first rotator position to a second rotator position by cooperation with the delivery member cover 21. The rotator 37 has external cam surfaces 37a configured to cooperate with a respective one of the radially inwards extending protrusions 21b of the delivery member cover 21. When the delivery member cover 21 is moved linearly from the extended position to the retracted position, the radially inwards extending protrusions 21b cooperate with the respective cam surface 37a, which causes the rotator 37 to rotate from the first rotator position to the second rotator position. The flexible arms 39a are thereby allowed to flex radially outwards into recesses or windows in the inner surface of the rotator. The plunger rod 29 is thereby released form its initial axial position.

In use, the cap 1 is first removed from the housing 19, which causes the removal of the delivery member shield 13 from the delivery member 25a and exposing of the delivery member. The medicament delivery device 17 is then placed onto the injection site with the delivery member cover 21 facing the injection site. Next, the delivery member device 17 is pressed towards the injection site, forcing the delivery member cover 21 from the extended position to the retracted position, which triggers medicament delivery as described above.

During assembly of the medicament delivery device 17, the cap 1 may in a step a) be attached to the proximal housing end 19a. Next, in a step b) the medicament container 25 with the delivery member shield 13 provided on the delivery member 25a is arranged or placed in the housing 19 via a distal opening of the housing 19. The power pack may according to one example be arranged in the housing 19 in step b) with the medicament container 25, because the power pack may be assembled with the medicament container 25. Alternatively, the power pack may be placed in the housing in a step c) after step b).

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the injection assemblies described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

The inventive concept has mainly been described above with reference to a few examples. However, as is readily appreciated by a person skilled in the art, other embodiments than the ones disclosed above are equally possible within the scope of the inventive concept, as defined by the appended claims.

1-14. (canceled)

15. A cap for a medicament delivery device including a delivery member shield, wherein the cap comprises:
a body having a proximal end portion and a distal end portion, wherein the distal end portion has a smaller radial dimension than the proximal end portion,
the distal end portion having a central channel configured to receive the delivery member shield,
wherein the distal end portion comprises a plurality of flexible first clips and a plurality of second clips extending radially inwards into the central channel, wherein each second clip extends further in a proximal direction towards the proximal end portion than the first clips.

16. The cap of claim 15, wherein each first clip is semi-circular in a circumferential direction of the cap.

17. The cap of claim 15, wherein the second clips are pointy.

18. The cap of claim 17, wherein the second clips comprise two proximally directed tips.

19. The cap of claim 18, wherein the tips of each second clip are separated by a notch.

20. The cap of claim 15, wherein the distal end portion comprises distally extending arms, wherein each first clip is connected to two, in the circumferential direction of the cap, adjacent arms, and each second clip is connected to two, in the circumferential direction of the cap, adjacent arms.

21. The cap of claim 20, wherein each arm is connected to one first clip and one second clip.

22. The cap of claim 15, wherein the cap is a single integral part.

23. The cap of claim 15, wherein the cap is made of plastic.

24. The cap of claim 15, wherein each first clip has a first clip distal end and each second clip has a second clip distal end, wherein along a longitudinal axial direction of the cap, the first clip distal ends and the second clip distal ends are level.

25. A medicament delivery device comprising:
a housing;
a medicament container arranged in the housing, the medicament container including a delivery member;
a delivery member shield covering the delivery member; and
a cap as claimed in any of the preceding claims, wherein the central channel of the distal end portion receives the delivery member shield.

26. The medicament delivery device of claim 25, wherein the first clips bear against a distal end of the delivery member shield.

27. The medicament delivery device of claim 25, wherein the delivery member shield comprises a plurality of radial shield windows, wherein the second clips each extend into a respective shield window of the plurality of radial shield windows.

28. A method of assembling the medicament delivery device of claim 25, comprising:
a) attaching the cap to a proximal housing end of the housing; and
b) arranging the medicament container in the housing via a distal opening of the housing, such that the delivery member shield is received by the distal end portion of the cap.
